(19) United States
(12) Patent Application Publication
(10) Pub. No.: US 2022/0040417 A1
(43) Pub. Date: Feb. 10, 2022

(54) Nocireceptor Blockade

(71) Applicant: Todd O'Brien, Orono, ME (US)

(72) Inventor: Todd O'Brien, Orono, ME (US)

(21) Appl. No.: 17/397,804

(22) Filed: Aug. 9, 2021

Related U.S. Application Data
(60) Provisional application No. 63/062,960, filed on Aug. 7, 2020.

(51) Int. Cl. A61M 5/42; A61M 19/00; A61H 23/02; A61F 7/00

(52) U.S. Cl. CPC A61M 5/422; A61M 19/00; A61H 23/02; A61M 2205/586; A61F 2007/0063; A61H 2201/105; A61F 7/00

(57) ABSTRACT

Systems, Methods and Devices to apply concomitant cold and vibratory stimulus immediately prior to injection to reduce pain, in a simple and mechanically advantaged device actuatable by a user's non-dominant hand or a third party.

This application claims the full Paris Convention benefit of, and priority to, U.S. Provisional Patent Application Ser. No. 63/062,960, filed Aug. 7, 2020.

### FIELD OF THE INVENTION

The present invention is designed to be a handheld instrument generally operated with the user's non-dominant hand while administering an injection with the dominant hand.

### BACKGROUND OF THE DISCLOSURES

The use of vibration and cold to reduce the pain of needlesticks during injections is well established. Cold is known to stimulate C-fiber nerve receptors which are also responsible for pain perception. These nerves known as nociceptors are less sensitive to painful stimuli during cold exposure.

Cold applied in a variety of methods including sprays, ice and frozen gel packs is often used in this way to temporarily reduce pain perception during injections. The effect of vibratory stimulus on injection pain has a less direct mechanism.

Mechanoreceptors, particularly Meissner's and Pacinian corpuscles, are most sensitive to vibrations in the range of 10-300 Hz. According to the gate theory of pain, when these vibration sensitive receptors are stimulated, afferent signals are sent to the spinal cord where they are thought to reduce or interfere with the transmission of afferent pain signals to the central nervous system.

In effect, when applied immediately prior to and during an injection, vibration is thought to result in a partial, transient blockade of nociceptive signals thereby reducing patient pain perception.

Prior Art: U.S. Pat. No. 8,449,482 Blaine May 2013; U.S. Pat. No. 8,740,960 Baxter June 2014; U.S. Pat. No. 9,439,829 Lee September 2016; and, U.S. Pat. No. 9,575,766 Goldberg June 2017;
Other publications include: Sato M. “Response of Pacinian Corpuscles to Vibration.” J. Physiol, 159, p. 391-409, 1961.
Hollins, M., et al., “Pacinian Signals Determine the Direction and Magnitude of the Effect of Vibration on Pain.” Perception 0 (0); 1-13, 2017;
Hollins, M., et al., “How does vibration reduce pain?” Perception, 43; 70-84, 2014;
Hollins, M., et al., “Vibratory Antinociception: Effects of Vibration Amplitude and Frequency.” Journal of Pain, 4(7):381-391, 2003;
Leung Y. et al. “Time-Course of Vibratory Adaptation and Recovery in Cutaneous Mechanoreceptive Afferents.” J Neurophysiol 94(5): 3037-3045, 2005.
Shanna P. et al., “Investigating the Efficacy of Vibration Anesthesia to Reduce Pain from Cosmetic Botulinum Toxin Injections.” Aesthetic Surgery Journal 31 (8); 966-971, 2011.
Ching D, et al., Effect of the DentalVibe injection system on pain during local anesthesia injections in adolescent patients.” Pediatric Dentistry 36(1):51-5, 2014.
Pasterczyk-Szczurek A., et al., “Parameters of vibration stimulation for the relief of pain of different origins and locations.” Medical Rehabilitation 22 (2):20-29, 2018.
Kakigi, R, et al., “Mechanisms of pain relief by vibration and movement.” J Neurology, Neurosurgery, and Psychiatry 55:282-286, 1992.
Manfredi, L., et al., “The Effect of Surface Wave Propagation on Neural Responses to Vibration in Primate Glabrous Skin.” PLoS ONE 7(2): e31203. doi:10.1371/journ (2012).

### BRIEF DESCRIPTION OF THE DRAWINGS

Various preferred embodiments are described herein with references to the drawings in which merely illustrative views are offered for consideration, whereby:

FIG. 1 is a top view of a handheld instrument according to the present disclosures;

FIG. 2 is a side view of a handheld instrument according to the present disclosures; and

FIG. 3 is a top partial plan view of the disclosed handheld instrument during operation.

### DETAILED DESCRIPTION OF THE DISCLOSURES

The present invention is designed to be a handheld instrument generally operated with the user's non-dominant hand while administering an injection with the dominant hand. Alternatively, an assistant may operate the device with the injection is administered.

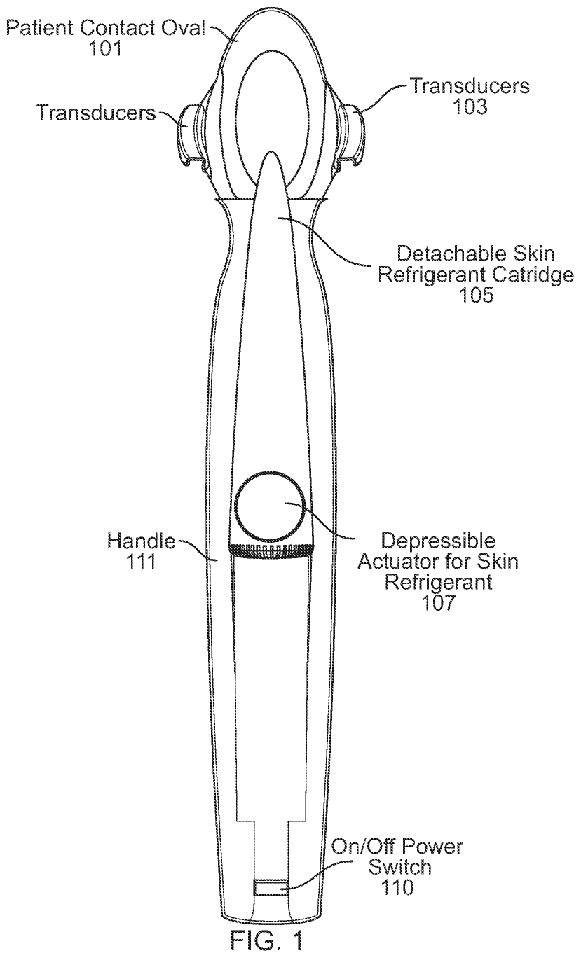

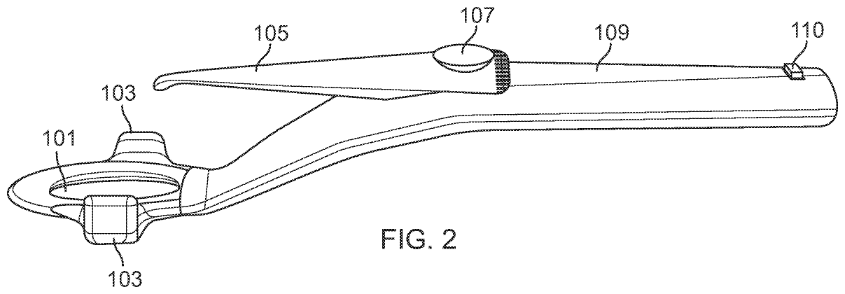

Artisans understand the Figures are schematic/cartoons and the actual shapes of prototype are not depicted to scale. Referring now to FIGS. 1-3, handle 111 section of the device includes a power supply, printed circuit board, on/off switch 110, and integrated refrigerant cartridge 105.

At least two transducers 103 capable of vibrating in the range of 10-300 Hz are located on opposing sides of a generally oval shaped patient contact component 101 extending off the handle section 111.

These two transducers 103 vibrate in phase and are spatially oriented such that their waves collide in the skin in the center of the oval shaped patient contact component 101. Through the phenomenon of constructive interference, these waves combine to form a larger amplitude wave 317.

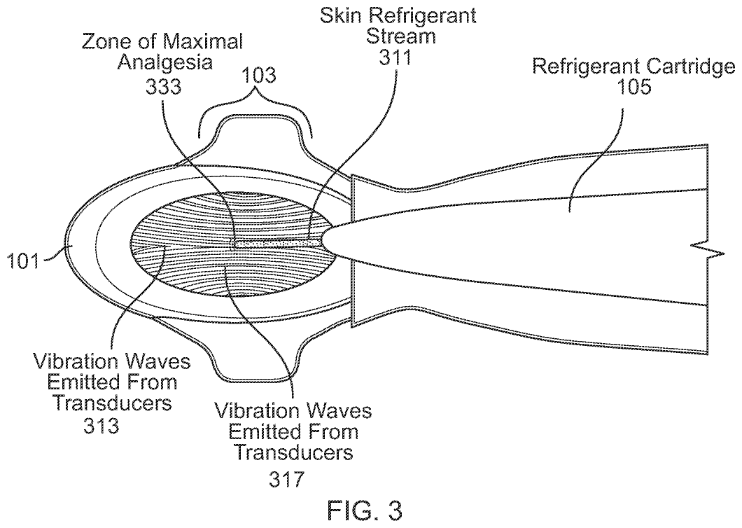

Referring now to FIG. 3, area of maximal vibration amplitude 313 provides optimal analgesia 333 during injection. Integrated, detachable cartridge 105 of skin refrigerant such as ethyl chloride is attached to the handle component 111 of the device.

Cartridge 105 includes a depressible mechanical actuator 107 that initiates a spray of skin refrigerant 311 while depressed. The spray discharged from cartridge 105 forms a coherent stream 311 until skin contact.

Stream 311 pinpoints the zone of maximum vibration amplitude 333 thereby providing guidance for needle placement during injection. Electronic control of transducers 103 is provided by a printed circuit board with integrated microprocessor.

Custom waveform 317 providing maximal stimulation of those mechanoreceptors sensitive to vibration is stored in the programmable microprocessor. This waveform generally between 10-300 Hz consists of sine waves in a modified sweep configuration.

The disclosed invention provides optimal transient analgesia to the skin through targeted, simultaneous stimulation of cold-sensitive C-fiber nerves and vibration-sensitive mechanoreceptors thereby reducing patient skin perception during injection.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially-feasible embodiment are often not depicted in order to facilitate a less obstructed view of these various embodiments of the present invention.

### DETAILED DESCRIPTION OF THE INVENTION

The present inventor has developed a novel medical device (Coolvibe™) type of topical pre-injection analgesia device, (O'Brien Medical, Maine, USA) to overcome issues regarding injections, which may include vaccinations, and the anxiety and significant pain imparted to patients thereby. Since vibrations and cold application are known to reduce pain of injection, the instant solution is believed to constitute progress in science and the useful arts.

As described below and herein, the present inventions include vibration and cold in a novel, user-friendly, ergonomic and elegantly simple form. Namely, custom waveforms have been developed to maximally stimulate vibration sensitive nerve receptors. Constructive interference results from relative orientation of these subject vibration sources, which increases vibration amplitude. According to the process and claimed methods of treatment below, vapocoolant skin refrigerant is supplied via integrated disposable cartridge which directs a vapocoolant stream at the area of maximal vibration amplitude. Stream contacts skin at area of maximal topical analgesia targeting optimal needle placement.

Use of the device is as follows: Referring now to the self-explanatory and reference designated figures:

The power button on the handle is actuated.

The patient contact component is gently placed on the skin over the intended injection site.

The skin refrigerant cartridge is actuated for about 10 seconds, resulting in a coherent refrigerant stream identifying the area of most combined analgesic effect.

Injection is performed in the targeted area.

Device is powered off and cleaned for re-use.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially-feasible embodiment are often not depicted in order to facilitate a less obstructed view of these various embodiments of the present invention

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled. steps are indicative of one embodiment of the presented method. Other steps and methods may be conceived that are equivalent 111 function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed—some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using, consisting of, or consisting essentially of language. When used in the claims, whether as filed or added per amendment, the transition term “consisting of” excludes any element, step, or ingredient not specified in the claims. The transition term “consisting of” essentially limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the itivemion includes a tangible, non-transitory computer readable medium for memory. Exemplary devices for use as memory include semiconductor memory devices, (e.g., EPROM, EEPROM, solid state drive (SSD), and flash memory devices, (e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for effecting transient nociceptor blockade, which comprises, in combination:
at least a patient contact oval;
transducers disposed about the contact oval;
a detachable cartridge for dispensing skin refrigerant equipped with a depressable actuator for skin refrigerant;
a handle; and,
an on/off power switch.

2. The system of claim 1, ergonomically arrayed within a device which is lightweight and operable with one hand of a user for administration to a patient.

3. The system of claim 2, where in the detachable cartridge for dispensing skin refrigerant is positioned to deliver a stream of skin refrigerant to an area equidistant from the transducers during operation.

4. The system of claim 3, whereby vibration waves emitted from the transducers, in combination with the skin refrigerant stream, create a zone of maximal analgesia at a central position within the patient contact oval.

5. The system of claim 4, whereby a user may operate the device as a handheld instrument with the user's non-dominant hand, in order to administer an injection with the dominant hand.

6. The system of claim 4, whereby an assistant may alternatively operate the device while an injection is administered.

7. The system of claim 5, wherein the handle section of the device includes a power supply, a printed circuit board, and an on/off switch and integrated refrigerant cartridge.

8. The system of claim 7, where the transducers are capable of vibrating in the range of 10-300 Hz.

9. The system of claim 8, wherein said transducers are located on opposing sides of a generally oval shaped patient contact component extending off the handle section.

10. The system of claim 9, whereby the transducers vibrate in phase and are spatially oriented such that their waves collide in the skin in the center of the oval shaped patient contact component.

11. The system of claim 10, whereby the vibration in phase combines to form a larger amplitude wave, via constructive interference.

12. The system of claim 11, whereby optimal analgesia during injection is established.

13. The system of claim 12, the integrated, detachable cartridge containing a skin refrigerant such as ethyl chloride.

14. The system of claim 13, said cartridge including a depressable mechanical actuator which initiates a spray of skin refrigerant while depressed.

15. The system of claim 14, wherein the spray discharged from the cartridge forms a coherent stream until skin contact.

16. The system of claim 15, whereby the stream pinpoints the zone of maximum vibration amplitude thereby providing guidance for needle placement during injection.

17. The system of claim 16, whereby electronic control of the transducers is provided by a printed circuit board with integrated microprocessor housed within the handle of the device.

18. The system of claim 17, whereby a custom waveform providing maximal stimulation of the mechanoreceptors sensitive to vibration is stored in the programmable microprocessor, said waveform being between at least about 10 and 30 Hz, consisting of sine waves in modified sweep configuration.

19. A method of providing optimal trasient analgesia to the skin through targeted, simultaneous stimulation of cold-sensitive C-fiber nerves and vibration-sensitive mechanoreceptors; comprising, in combination, the steps of:
providing a handheld instrument/device wherein the handle section includes a power supply, printed circuit board, on/off switch and integrated refrigerant cartridge; and,
at least two transducers arrayed about a patent contact oval;
actuating the skin refrigerant cartridge for at least about 10 seconds, resulting in a coherent refrigerant stream identifying the area of most combined analgesic effect;
injecting patent in targeted area;
powering off device and cleaning for next use.

20. The method of claim 19, operated by at least one modality selected from the group of by the user's non-dominant hand while administering the injection with the dominant hand; and having an assistant operate the handheld instrument/device while injection is administered.
